SEQUENCE LISTING (3 sequences)

SEQ ID NO: 1 - DNA, 21 bp, Artificial Sequence
  Synthetic Construct
gagtcctgcatcagccaagag

SEQ ID NO: 2 - DNA, 22 bp, Artificial Sequence
  Synthetic Construct
ggatccaccttttctgcttgac

SEQ ID NO: 3 - DNA, 26 bp, Artificial Sequence
  Synthetic Construct
tgttcccaagtggagccaaccaaact

(19) United States
(12) Patent Application Publication
(10) Pub. No.: US 2021/0371905 A1
(43) Pub. Date: Dec. 2, 2021

(54) SURVEILLANCE METHOD TO SCREEN ASYMPTOMATIC ESSENTIAL WORKERS FOR EXHALATION OF SARS-COV-2

(71) Applicant: VANDERBILT UNIVERSITY, Nashville, TN (US)

(72) Inventor: Frederick R. Haselton, Nashville, TN (US)

(21) Appl. No.: 17/331,151

(22) Filed: May 26, 2021

Related U.S. Application Data
(60) Provisional application No. 63/030,002, filed on May 26, 2020.

(51) Int. Cl. C12Q 1/686; A41D 13/11; C12Q 1/70

(52) U.S. Cl. CPC C12Q 1/686; A41D 2300/20; C12Q 1/70; A41D 13/11

(57) ABSTRACT

Disclosed herein is a COVID-19 surveillance method for detecting exhaled virions trapped within used face masks. This is a surveillance and warning system for identifying asymptomatic and pre-symptomatic individuals, particularly essential workers required to wear masks while at work. This can include healthcare workers, first responders, nursing home personnel, postal workers, or employees at meat packing and other production facilities. A piece of filter paper can be added to the inside of a standard face mask, which can be removed at the end of a shift. Mask inserts from a group of employees can be pooled and tested using standard RT-PCR for virions collected during normal exhalation over the time the mask is worn. As envisioned, if the group test is positive, additional follow-up or contact tracing could be initiated to identify the individual or individuals requiring treatment or quarantine.

### CROSS-REFERENCE TO RELATED APPLICATIONS

This application claims benefit of U.S. Provisional Application No. 63/030,002, filed May 26, 2020, which is hereby incorporated herein by reference in its entirety.

### STATEMENT REGARDING FEDERALLY SPONSORED RESEARCH OR DEVELOPMENT

This invention was made with Government Support under Grant No. A1135937 awarded by the National Institutes of Health. The Government has certain rights in the invention.

### SEQUENCE LISTING

This application contains a sequence listing filed in electronic form as an ASCII.txt file entitled “222230-1040 Sequence Listing_ST25” created on May 24, 2021 and having 870 bytes. The content of the sequence listing is incorporated herein in its entirety.

### BACKGROUND

As of May 7th, there have been 3.77 million confirmed cases of COVID-19 worldwide resulting in 264,000 reported disease-induced deaths. The US Centers for Disease Control and Prevention (CDC), based on an assembled collection of forecast models, predicts the United States alone to surpass 100,000 COVID-19 related deaths by June 1st (cdc.gov). The severity of the pandemic has caused and will continue to cause significant national and global economic disruption. Due to fear-induced individual behavior changes, along with the necessary responses to mitigate the spread such as government enforced “stay-at-home” orders, the COVID-19 pandemic raises concerns for impending economic recessions in countries which make up the world's largest economies, such as the US (Nicola, et al. (2020) Int J Surg. April 16). The disease-induced impact permeates virtually all sectors of the economy: primary (agricultural and oil), secondary (manufacturing industry), and tertiary (education, the finance industry, healthcare and the pharmaceutical industry, technology research and development) sectors (Nicola, et al. (2020) Int J Surg. April 16). Dramatic statistics reported by the U.S. Department of Commerce and the Congressional Budget Office (CBO) portray the widespread affect—U.S. unemployment claims have reached approximately 33.5 million in just the last seven weeks and the CBO projects a real gross domestic product (GDP) decline at an annual rate of about 40 percent for the second quarter of 2020.

Although conclusive evidence has not been reported, COVID-19 primarily appears to be spread from person to person via small respiratory droplets that are expelled from a person's nose our mouth through sneezes, coughs, singing, speaking and even normal exhalation. The virus-containing droplets may land on an individual, surfaces that an individual interacts with, or may be in an aerosolized form and subsequently be inhaled causing infection. Studies suggest that the severe acute respiratory syndrome coronavirus 2 (SARS-CoV-2) virus can remain stable for days on surfaces such as plastics, cardboards, and stainless steels and for as long as 3 hours in the air (van Doremalen, N., et al. (2020) N Engl J Med 382:564-1567). Airborne transmission studies of SARS-CoV-2 are contradictory (van Doremalen, N., et al. (2020) N Engl J Med 382:564-1567; Cheng, V C C., et al. (2020) Infect Control Hosp Epidemiol 41:493-498; Ong, S W X., et al. (2020) JAMA. 2020 Mar. 4; Lewis, D. (2020) Nature 580:175), but knowledge about the mechanisms of similar viruses suggest that airborne transmission of SARS-CoV-2 is likely under some conditions. Its predecessor, SARS-CoV-1 has been shown to spread in the air. Studies of the pathway of SARS transmission in Hong Kong's Prince of Wales Hospital (6,7) as well as in healthcare facilities in Toronto, Canada (Booth, T F., et al. (2005). J Infect Dis 191:1472-1477) and concluded that the main mechanism of virus transmission was airborne. It has also been shown that aerosol transmission accounts for approximately half of Influenza A transmission (Cowling, B J., et al. (2013) Nat Commun 4:1935), and another recent study has established that influenza virus can be emitted by breathing or speaking, without even coughing or sneezing (Yan, J., et al. (2018) Proc Natl Acad Sci USA 115:1081-1086). Considering the similarities between SARS-CoV-2 and these respiratory viruses, it is highly likely that the novel coronavirus also spreads by air.

In addition to concerns of airborne transmission, it appears that asymptomatic individuals can also spread the virus. A literature review performed by the CDC assessed peer review articles from January 2020-April 2020 focusing on pre-symptomatic and asymptomatic SARS-CoV-2 transmission. Upon completion of this literature review on community spread of COVID-19 the CDC began recommending that people wear cloth face coverings to reduce exhalation of aerosolized virus (Furukawa, N W., et al. (2020) EID Journal 26(7)). A German business man exposed to a colleague visiting from China inadvertently exposed two other colleagues while he was pre-symptomatic and those colleagues subsequently tested positive (Rothe, C., et al. (2020) N Engl J Med 382:970-971). Four reports documented SARS-CoV-2 RNA with lower Ct values, which is indicative of a higher concentration of viral RNA, in samples from persons who never developed symptoms (Hoehl, S., et al. (2020) N Engl J Med 382:1278-1280; Kam, K Q., et al. (2020) Clin Infect Dis. February 28; Le, TQM. (2020) EID Journal 26(7); Zou, L., et al. (2020N Engl J Med 382:1177-1179). Two reports described specimens with low Ct values among pre-symptomatic and asymptomatic residents of a nursing home (Arons, M M., et al. (2020) N Engl J Med. April 24; Kimball, A., et al. (2020) MMWR Morb Mortal Wkly Rep 69:377-381). Among these reports Ct values for SARS-CoV-2 RNA in asymptomatically infected persons ranged from 14-40 (Hoehl, S., et al. (2020) N Engl J Med 382:1278-1280; Kam, K Q., et al. (2020) Clin Infect Dis. February 28; Le, TQM. (2020) EID Journal 26(7); Zou, L., et al. (2020N Engl J Med 382:1177-1179; Arons, M M., et al. (2020) N Engl J Med. April 24). A study on pre-symptomatic infected patients reported an average Ct 24 (range 15-38) (Arons, M M., et al. (2020) N Engl J Med. April 24). Two reports described culture of infectious virus from persons who were asymptomatic (Hoehl, S., et al. (2020) N Engl J Med 382:1278-1280) and pre-symptomatic (Arons, M M., et al. (2020) N Engl J Med. April 24). They did not identify actual transmission, however the low RT-PCR Ct values and ability to isolate infectious virus provide evidence that SARS-CoV-2 transmission probably occurs in persons not demonstrating symptoms.

The World Health Organization has outlined and continues to update national and global strategic plans for pandemic control. These plans include proactively engaging and mobilizing communities to limit exposure, finding, testing, isolating, and caring for COVID-19 cases and their contacts, maintaining essential health services and clinical care to reduce mortality, and adapting these strategies based on a specific locale's risk, capacity, and vulnerability (WHO. (2020) COVID-19 Strategy Update). WHO recommends suppression of community transmission through a variety of personal and community level measures. Personal measures include reducing the risk of person-to-person transmission by hand washing, physical distancing, and respiratory etiquette, which includes the use of face masks for aerosol and droplet containment. Community-level measures include social distancing practices that prevent mass gatherings, closing nonessential businesses and educational institutions, and reducing the use of mass transport, including local public, national, and international travel, and ensuring protection of health care workers and vulnerable groups (including high risk individuals, patients in assisted living facilities, etc.) by providing correct personal protective equipment. Application of these policies can be seen throughout national and international news (WHO. (2020) COVID-19 Strategy Update). Use of these measures in Hubei province further demonstrates the effectiveness of these interventions (Tian, H., et al. (2020) Science 368:638-642). Studies also indicate that face coverings capture SARS-CoV-2 virus and may aid transmission reduction (Fineberg, H. V. (2020) Rapid Expert Consultation on the Possibility of Bioaerosol Spread of SARS-CoV-2 for the COVID-19 Pandemic. The National Academies Press, Washington, D.C.).

With respect to workplace changes, the Occupational Safety and Health Administration under the U.S. Department of Labor has published guidance on preparing workplaces, both essential and nonessential, for COVID-19 (Labor, U D. Et al. (2020) Guidance on preparing workplaces for COVID-19). Businesses are to develop and infectious disease preparedness plan, which includes assessing where, how, and what sources SARS-CoV-2 might result in worker exposure. Businesses are guided to implement basic infection control and safe work practices including frequent and thorough hand washing (or sanitation with at least 60% alcohol), respiratory etiquette, use of personal protective equipment (PPE such as use of gloves, masks, and eye coverings), and maintenance of routine housekeeping practices (disinfection of surfaces and equipment in the work environment using an EPA-approved disinfectant). Suggested OSHA engineering controls, such as implementing physical barriers (e.g. sneeze guards) and using only drive-thru for services when possible have been widely used throughout the COVID-19 outbreak. Administrative controls to minimize contact between workers and clients include delivery-only service at restaurants, daily temperature checks and symptom screenings for employees, and required use of risk-level appropriate PPE (Labor, U D. Et al. (2020) Guidance on preparing workplaces for COVID-19).

### SUMMARY

Disclosed herein is a kit for viral surveillance of essential workers. In some embodiments, the kit contains filter paper inserts configured to attach or adhere to the inside of a face mask. In some embodiments, the kit further contains extraction buffers configured to extract virion RNA from the filter paper inserts. In some embodiments, the kit further contains reagents for reverse transcription of the virion RNA into cDNA. In some embodiments, the kit further contains oligonucleotide primers and/or probes configured to assay for the presence of the cDNA. In some embodiments, the kit further contains oligonucleotide primers and/or probes configured to assay for the presence of a bacteria resident in normal oral cavity. In some embodiments, the kit further contains reagents for reverse transcription of

Also disclosed herein is a surveillance face mask with one or more removable filter paper inserts attached or adhered to the inside of the face mask. In some cases, the filter paper insert is a water-soluble fabric, such as a polyvinyl alcohol (PVA) fabric.

Also disclosed herein is a method for surveillance of a group of subjects, such as essential workers, the method involving providing face masks to the subjects, the face masks having filter paper inserts attached or adhered to the inside of the face masks, the filter paper inserts from the face masks after use, pooling them together, and assaying the pooled filter paper inserts for virion RNA by RT-PCR.

In some embodiments, the method further involves screening the group of subjects for infection if respiratory virus RNA is detected in the pooled filter paper inserts.

In some embodiments, the method further involves assaying the filter paper inserts for a bacteria or virus resident in normal oral cavity. There are over 700 separate types of bacteria commonly found in the human mouth. Streptococci make up a large part of oral bacteria. There are four main species within Streptococci: the mutans, salivarius, anginosus, and mitis groups. In some embodiments, the bacteria is Streptococcus mitis. In some cases, the virus is a herpesvirus. In some cases the virus is a cytomegalovirus (CMV).

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a scheme for measuring Sars-Cov-2 virions captured in a face mask. Not envisioned as another diagnostic for an individual, but more for a pooled sample test using masks worn for long periods and batch-processed at the end of every day. A positive group could be instructed to seek follow-up testing.

FIGS. 2A and 2B show embodiments of a disclosed surgical mask containing paper inserts.

FIG. 3 shows initial extraction of irradiated virus from two types of Whatman filter paper. Recovery for 10⁶, 10⁶, and 10³ copies for Whatman 3MM chromatography paper (left two bars) and Grade 4 (right 3 bars). Data is normalized to recovery from an unspotted sample.

FIG. 4 shows recovery of SARS-CoV-2 from polyvinyl alcohol material. Comparison of irradiated SARS-CoV-2 RNA recovery from polyvinyl alcohol after inactivation treatments and spin column extraction using anti-viral lysis buffer (AVL) or 62° C. dry heat. Also shown direct spike of SARS-CoV-2 into RT-PCR (Direct Spike) and extraction without treatment (Control).

FIG. 5 shows collection and extraction capabilities of mask insert when directly spiked with gamma-irradiated SARS-CoV-2. This example assesses the performance of the mask insert to collect and retain irradiated SARS-CoV-2 virus for extraction and detection. The figure shows successful capture and extraction of gamma-irradiated SARS-CoV-2 (BEI Reagents). These studies were used to determine the effectiveness of the mask insert (yellow) collection strategy as compared to a control consisting of direct sampling of gamma-irradiated virus in solution (red). The small difference in cycle threshold (Ct) for amplification demonstrates minimal loss of sample during collection and extraction during the mask insert processing protocol. To ensure the integrity of the study conducted, a no template control (NTC) was included in the PCR run.

FIG. 6 shows employment of the mask collection strategy for collection and extraction of exhaled human RNase P from exhaled samples. Measures of sample integrity are often used to assess testing performance in diagnostics. The mask collection strategy was used to determine the effectiveness of human RNase P as a biomarker for exhaled breath collection and as a potential marker of sufficient sample. The approach, which utilizes RT-PCR for detection, successfully detected the RNA for RNase P on a mask insert worn for 30 minutes inside a clean surgical mask. RNase P abundance yielded from the strategy was compared to a saliva sample taken from the same human subject. To ensure the integrity of the study conducted, a no template control (NTC) was included in the PCR run.

FIG. 7 shows identification of model exhaled oral commensal bacteria target and measurement of the presence of candidate in exhaled breath. Exhaled breath from healthy volunteers contains bacteria similar to microorganisms that cause respiratory illness. Using this mock sample approach, combined with the mask requirement for laboratory personnel, we optimized droplet capture using a model system based around a common commensal bacteria found in the oral cavity. There are more than 700 bacterial species in the oral cavity with Streptococcus mitis is the most commonly found species. The preliminary data shown here used published primers and probes and illustrates, for one individual, the relative copies of s. mitis found in a saliva sample compared to those extracted from a mask-insert. Extraction was performed using a modification of magnetic bead extraction techniques.

FIG. 8 shows exhalation time characteristics via collection and extraction from a mask insert. In this example, exhalation characteristics are observed via the mask collection strategy. Exhalation and capture studies including but not limited to time studies (figure), aerosolization studies (e.g., singing vs. talking vs. breathing), and variability studies may be investigated to determine the best time and strategy for aerosol droplet collection. In the figure, two separate mask inserts were worn in clean surgical masks to test the variation in sample collection when worn for 15 minutes (red) and 120 minutes (blue). Amplification of both time-study masks demonstrates successful detection of human subject exhaled S. Mitis. Both samples were detected between the third and fourth orders of magnitude. Lower cycle threshold (Ct) for amplification of the 15 minute mask insert as compared to the 120 minute mask insert demonstrates complexity in human exhalation characteristics. To ensure the integrity of the study conducted, a no template control (NTC) was included in the PCR run.

FIG. 9 shows example pooling methods. There was little change in copies-detected when an s. mitis spiked mask-insert was processed individually or when combined and processed with 10 other unspiked inserts. In a test of the pooling strategy, we showed that pooling of inserts does not reduce detectability relative to individual processing. PCR cycle number is shown on the x-axis and relative fluorescence on the vertical axis. Standards labeled 10² copies/uL and 10⁴ copies/uL are shown. The line labeled 1 insert shows the signal for one mask insert spiked with s. Mitis target and the line labeled “1 Pooled with 10” indicates the signal when 9 negative inserts and combined with a single positive insert. Here, nucleic acid extraction was performed using a modification of a previously described protocol. The approach allows for increasing the number of beads in the initial binding step in proportion to the total required volume for N mask-inserts.

FIG. 10 shows an example pooling methodology. Schematic of high-volume nucleic acid extraction method for concentrating nucleic acids from a large volume. In this method, step 1 inactivated a collection of mask inserts in an antiviral solution. In step 2 magnetic beads are added to bind nucleic acids present in the antiviral medium. In step 3, a magnet is used to capture magnetic beads in a steel wool matrix as the high volume of AVL is expelled into a waste container. Step 4 oscillates flow to increase capture. In Step 5, successive processing steps precipitate and then eventually release the nucleic acids into a small volume for RT-PCR testing.

Overall, two basic approaches could be utilized for pooling mask-pads: [1] process each insert individually and pool the extracted material before PCR or [2] pool these collection-pads before extraction and perform a single extraction before PCR. The former method represents a more traditional strategy and applies to recent Emergency Use Authorization COVID-19 tests (for nasopharyngeal specimens) promulgated by Quest and LabCorp. However, the latter method (depicted in in this figure) offers greater logistical impact, with additional condensation of experimental steps and high scalability. It is more readily deployed during the initial collection of specimens in workplace environments (i.e. used pads pre-bundled into defined cohorts). Ultimately, we believe this approach would be most actionable for real-world utilization.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

Disclosed herein is a way to take advantage of the workplace expectation for essential workers to wear masks in the workplace, the property of masks to capture exhaled droplets, and available methods for pooled processing to identify an infectious group at the workplace for quarantine and testing follow-up. As disclosed herein, a collection material can be added to the inside of the mask to serve as a collection pad. In some embodiments, testing pooled masks has the advantage of saving costs and is currently in practice in many low resource settings. This embodiment can involve combining patient samples and runingn a single RT-PCR on the pooled sample. Pools that test positive are subsequently tested individually. These strategies are cost effective with minimal decrease in accuracy (Boobalaan, J., et al. (2019) Journal of Clinical Virology 117:56-60; Smith, D. M., et al. AIDS 23:2151-2158; van Schalkwyk, C., et al. (2019) BMC Infect Dis 19:136).

The disclosed devices and methods can be used to detect and monitor for any RNA virus, such as coronaviruses. Examples of (−)-strand RNA viral genera include arenaviruses, bunyaviruses, and mononegavirales. Species that are members of the arenavirus genus include, but are not limited to, are sabia virus, lassa fever virus, Machupo Virus, Argentine hemorrhagic fever virus, and flexal virus. Species that are members of the bunyavirus genus include, but are not limited, to hantavirus, nairovirus, phlebovirus, hantaan virus, Congo-Crimean hemorrhagic fever, and rift valley fever. Species that are members of the monoegavirales genus include, but are not limited to, filovirus, paramyxovirus, ebola virus, Marburg, and equine morbillivirus. Examples of (+)-strand RNA viral genera include, but are not limited to, picornaviruses, astroviruses, calciviruses, nidovirales, flaviviruses, and togaviruses. Species of the picornavirus genus include, but are not limited to, coxsackievirus, echovirus, human coxsackievirus A, human echovirus, human enterovirus, human poliovirus, hepatitis A virus, human parechovirus, and human rhinovirus. A species of the astrovirus genus, includes but is not limited to, human astrovirus. Species of the calcivirus genus include, but are not limited to, chiva virus, human calcivirus, and norwalk virus. Species of the nidovirales genus include, but are not limited to coronavirus and torovirus. Species of the flavivirus genus include, but are not limited to, Alfuy virus, Alkhurma virus, Apoi virus, Aroa virus, Bagaza virus, Banzi virus, Batu cave virus, Bouboui virus, Bukalasa bat virus, Bussliquara virus, Cacipacore virus, Carey island virus, Cowbone ridge virus, Dakar bat virus, Deer tick virus, Dengue virus type 1, Dengue virus type 2, Dengue virus type 3, Dengue virus type 4, Edge hill virus, Entebbe bat virus, Flavivirus sp., Gadgets gully virus, Hepatitis C virus, Iguape virus, Ilheus virus, Israel turkey meningoencephalitis virus, Japanese encephalities virus, Jugra virus, Jutiapa virus, Kadam virus, Kedougou virus, Kokobera virus, Koutango virus, Kunjin virus, Kyasanur forest disease virus, Langata virus, Louping III virus, Maeban virus, Modoc virus, Montana myotic leukoencephalitis virus, Murray Valley encephalitis virus, Naranjal virus, Negishi virus, Ntaya virus, Omsk hemorrhagic fever virus, Phnom-Penh bat virus, Potiskum virus, Powassan virus, Rio bravo virus, Rocio virus, Royal farm virus, Russian spring-summer encephalitis virus, Saboya virus, Saint Louis encephalitis virus, Sal vieja virus, San perlita virus, Saumarez reef virus, Sepik virus, Sitiawan virus, Sokuluk virus, Spondweni virus, Stratford virus, Tembusu virus, Tick-borne encephalitis virus, Tyulenly virus, Uganda 5 virus, Usutu virus, West Nile virus, and Yellow fever virus. Species of the togavirus genus include, but are not limited to, Chikugunya virus, Eastern equine encephalitis virus, Mayaro virus, O'nyong-nyong virus, Ross river virus, Venezuelan equine encephalitis virus, Rubella virus, and hepatitis E virus. The hepatitis C virus has a 5′-untranslated region of 340 nucleotides, an open reading frame encoding 9 proteins having 3010 amino acids and a 3′-untranslated region of 240 nucleotides. The 5′-UTR and 3′-UTR are 99% conserved in hepatitis C viruses.

Based on past studies, there seems to be significant evidence that SARS-CoV-2 airborne transmission material is available for detection. For example, it is known that the SARS-CoV-2 predecessor, SARS-CoV-1, was transmitted mostly via droplets in air and aerosols in indoor scenarios (Morawska, L., et al. (2020) Environ Int 139:105730). Droplet size tends to decrease with distance from the origin of emission, and higher concentrations of pathogen exist in larger droplets (Morawska, L., et al. (2020) Environ Int 139:105730). The spray of respiratory droplets has been seen to reach up to 8 meters away from the source during an uncovered violent sneeze (Bourouiba, L. (2020) JAMA. March). During their transmission in air, droplets continue to breakup into smaller drops (Scharfman, B E., et al. (2016) Exp Fluids 57:24). The largest droplets tend to settle onto surfaces within 1-2 m of the emission source (Bourouiba, L. (2016) N Engl J Med 375:e15). Also, due to the turbulence of a cough or sneeze, respiratory droplets in the center of the emission cloud may be shielded from the outside environment and therefore have a longer infectious life (Scharfman, B E., et al. (2016) Exp Fluids 57:24). The disclosed devices, kits, and methods allows for interception of these exhaled droplets on the inside of a face mask and testing these used masks for SARS-CoV-2 RNA.

The proposed method is very applicable to the requirement for essential workers to be able to go to work safely, and is uniquely positioned to identify groups of individuals for follow-up testing who may have the virus but are either asymptomatic or pre-symptomatic.

The air pocket within the mask maintains a higher humidity and temperature than the surrounding air. Therefore, droplets contained in the exhaled breath remain large enough in diameter size that they are efficiently trapped within the fibers of the face mask. Once the droplets are trapped within the mask during a normal use period (e.g. a shift), the used masks can be collected and dried in a microwave or in an autoclave in order to inactivate any infectious virions. The masks inserts can then be collected and pooled. RNA material can then be extracted from the mask inserts. Standard RT-PCR techniques can then be used to determine if virus biomarker is present. RT-PCR can have very high sensitivity and specificity to detect low levels of RNA. An additional advantage is that existing RT-PCR machines already in place could be used to perform this screening assay.

Another attractive feature of this pooled sampling approach is that it allows businesses which have many employees to test them as a group to determine if any one of them is infected with a candidate virus, such as SARS-CoV-2. Testing every employee every day would be cost prohibitive. However, with the pooled testing strategy, if a group tests negative the employer and employees are reassured. However, if the group tests positive, individuals within that group could have follow-up testing to make sure they are not in the initial stages of infection before returning to work. This strategy can potentially identify pre-symptomatic as well as symptomatic individuals who might not be self-reporting (Chow, E J., et al. (2020) JAMA. April 17).

An example embodiment of this technology is an absorbent pad, like a Whatman filter paper disk, that can be applied to the inside of a mask. This would allow collection of these paper disks into a solution for batch processing. Volume reduction strategies, such as HGMS magnetic beads (Pearlman, S I., et al. (2020) ACS Appl Mater Interfaces 12:12457-12467), would also be helpful if large volumes are required to recover material deposited onto the disks.

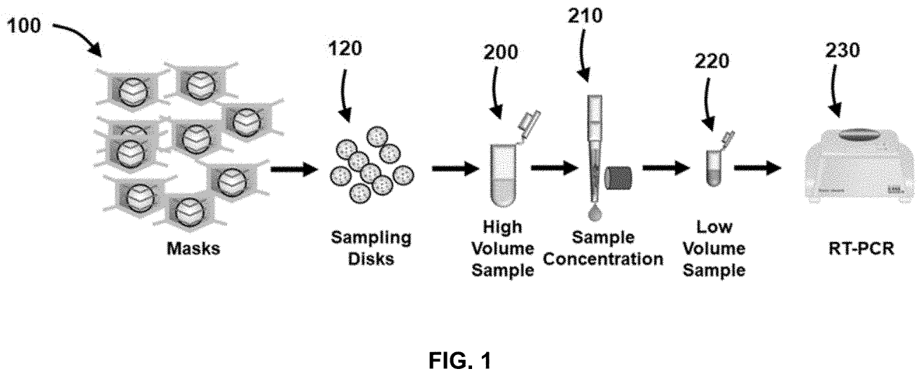

Turning now to FIG. 1, shown is a surveillance face mask 100 comprising removable filter paper inserts 120 attached or adhered to the inside of the face mask 110. The filter paper insert 120 is in some embodiments Whatman 4 Grade plain cellulose paper (Whatman Inc.), with the following manufacturer's specifications; particle retention greater than 20-25 μm, coarse porosity, filtration speed ASTM 12 sec., Herzberg 37 sec., and a smooth surface. Other similar filter materials and grades may be used include Whatman 3MM, Whatman 1, Whatman 3, Whatman 4, Whatman 6 and Pall 1660 membranes, Pall RSPJ037 Teflon membranes, and Sartorius Gelatin membrane filters (12602-37). In some embodiments, the filter material is an absorbent material that can be dissolved during the RNA extraction, such as a polyvinyl alcohol strip. In some embodiments, the filter material is any material that does not contain harmful chemicals that present hazard to the wearer, have high absorbency capability to retain condensed fluids over a long time period, do not contain RNase activity that breaks down viral RNA captured within the material, and preferably has low airflow resistance to maximize airflow through the insert materials.

In some embodiments, the removable filter paper inserts 120 are attached to the inside of the face mask 110 using a clip or fastener. In some embodiments, the removable filter paper inserts 120 are adhered to the inside of the face mask 110 by an adhesive or hook and loop material. For example, in some embodiments, the filter paper inserts 120 are attached to the inside of the face mask 110 by double-sided tape (preferably porous), a celco tab, a porous glue, or a staple. In some embodiments, the filter paper inserts 120 are built into the face mask 110 during manufacture.

The face mask 100 can be any commercially available face mask suitable for use by essential workers. The face mask 100 can be disposable or reusable. In some embodiments, the face mask 100 is a surgical mask made of a nonwoven fabric created using a melt blowing process. In some embodiments, the face mask 100 is an N95 mask. In some embodiments, the face mask 100 is an FFP1, FFP2, or FFP3 respirator mask.

The face mask 100 preferably has a sufficient number and/or surface area of filter paper insert 120 to collect a detectable amount of viral RNA. Factors that are expected to affect this include properties of the collection material itself, e.g. how much it absorbs per gram of material, the total surface area, how many inserts, what kind of air flow characteristics the insert material has and how does the air flow through the filter paper insert 120 match the surrounding air flow characteristics of the surgical mask. The area of the filter paper insert could be as small as a hole punch, e.g. approximately 3 mm in diameter, and could be as large as the entire area of the interior of the mask, e.g. added during manufacturing.

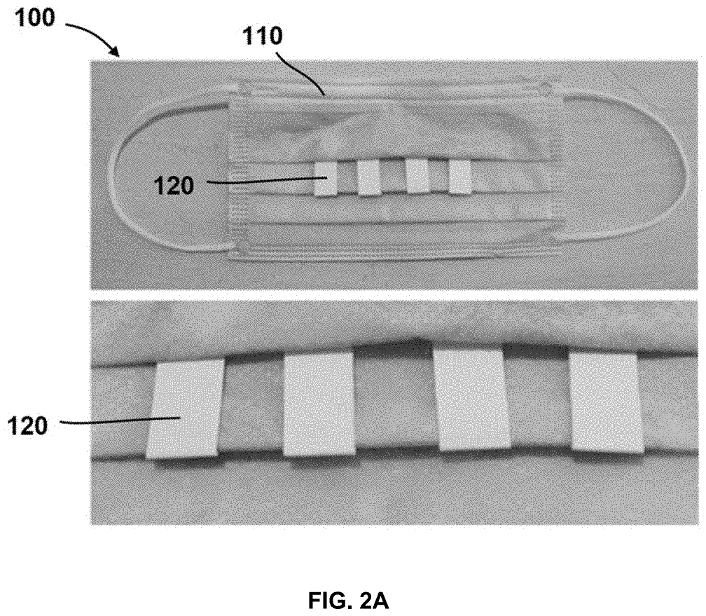

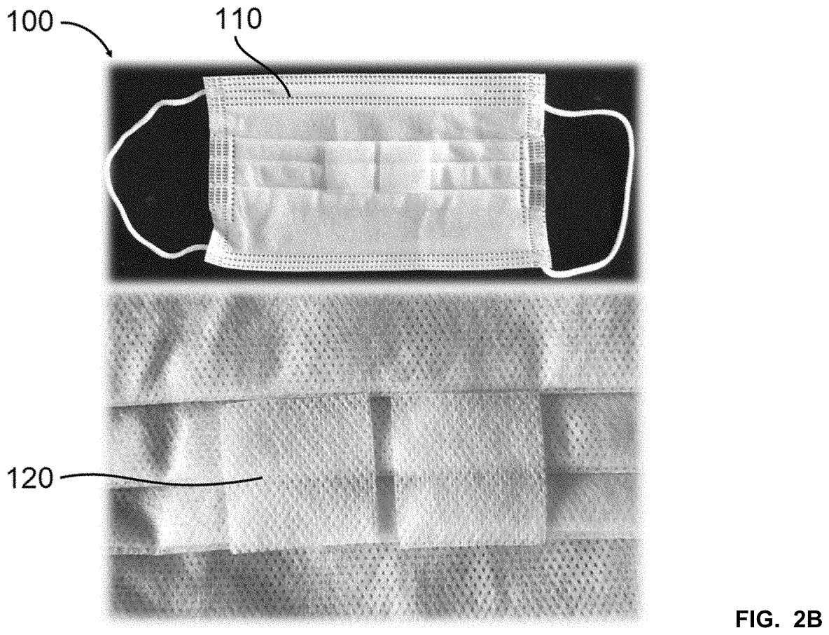

Shown in FIG. 2 is method for surveillance of essential workers, the method comprising: providing surveillance face masks 100 to each of the essential workers, the surveillance face masks 100 comprising filter paper inserts 120 attached or adhered to the inside of the face masks 110; removing and collecting the filter paper inserts 120 from the face masks 100 after use; and assaying the filter paper inserts 120 for virion RNA by RT-PCR 230.

The filter paper inserts 120 can in some embodiments be sterilized to kill any pathogens, such as virions, in the paper inserts 120. This can be done, for example, by heat or microwave radiation that dries the inserts and kills any virions. Standard commercial RNA extraction kits normally include a first solution designed to lyse and kill viruses. Normally this is done by chemical treatment using guanidine-isothiocyanate as is contained in Qiagen's RNeasy kit. This can also be done simply by boiling for approximately 10 minutes.

RNA present with the filter paper inserts 120 can then be extracted into a pooled sample volume 200 using routine methods. A number of techniques are known in the art, and several are commercially available (e.g., FormaPure nucleic acid extraction kit, Agencourt Biosciences, Beverly Mass., High Pure FFPE RNA Micro Kit, Roche Applied Science, Indianapolis, Ind.). RNA can be extracted from frozen tissue sections using TRIzol (Invitrogen, Carlsbad, Calif.) and purified using RNeasy Protect kit (Qiagen, Valencia, Calif.). RNA can be further purified using DNAse I treatment (Ambion, Austin, Tex.) to eliminate any contaminating DNA. RNA concentrations can be made using a Nanodrop ND-1000 spectrophotometer (Nanodrop Technologies, Rockland, Del.). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

Once the RNA is extracted, it can be amplified by reverse transcription-PCR (RT-PCR) for detection. In some embodiments, the sample volume can be reduced before RT-PCR to concentrate the RNA molecules in the sample. In some embodiments, the sample is purified and concentrated after reverse transcription of the RNA molecules into cDNA but prior to the PCR step. For example, a wide range of RNA extraction kits are commercially available and can be used in the disclosed devices and methods. In some embodiments, RNA extraction is accomplished using a magnetic bead method as described in Bordelon, H., et al. (2011) ACS Appl Mater Interfaces 3:2161-216; Bordelon, H., et al. (2013) PLoS One 8, e68369; and Pearlman, S. I., et al. (2020) ACS Appl Mater Interfaces 12:12457-12467, which are hereby incorporated by reference for these methods and reagents.

The extracted RNA can be analyzed using any suitable RT-PCR system, including real-time quantitative multiplex RT-PCR platforms and other multiplexing technologies such as GenomeLab GeXP Genetic Analysis System (Beckman Coulter, Foster City, Calif.), SmartCycler® 9600 or GeneXpert(R) Systems (Cepheid, Sunnyvale, Calif.), ABI 7900 HT Fast Real Time PCR system (Applied Biosystems, Foster City, Calif.), LightCycler® 480 System (Roche Molecular Systems, Pleasanton, Calif.), xMAP 100 System (Luminex, Austin, Tex.) Solexa Genome Analysis System (Illumina, Hayward, Calif.), OpenArray Real Time qPCR (BioTrove, Woburn, Mass.) and BeadXpress System (Illumine; Hayward, Calif.).

In alternative embodiments, the face mask is made of an absorbant material and is processed to remove a section of the mask for analysis.

In another embodiment, the present invention also provides kits for carrying out the methods described herein. For example, disclosed is a kit for viral surveillance of essential workers, the kit comprising: filter paper inserts 120 configured to attach or adhere to the inside of a face mask 100; extraction buffers configured to extract virion RNA from the filter paper inserts 120; reagents for reverse transcription of the virion RNA into cDNA; and oligonucleotide primers and/or probes configured to assay for the presence of the cDNA. In some cases, the kit also contains a positive control virion RNA or cDNA sample.

The kit may also comprise a sufficient quantity of reverse transcriptase, a DNA polymerase, suitable nucleoside triphosphates (including any of those described above), a DNA ligase, and/or reaction buffer, or any combination thereof, for the amplification processes described above. A kit may further include instructions pertinent for the particular embodiment of the kit, such instructions describing the primer pairs and amplification conditions for operation of the method. A kit may also comprise amplification reaction containers such as microcentrifuge tubes and the like. A kit may also comprise reagents for extracting RNA, including, for example, detergent.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Develop and Optimize Method for Collecting and Extracting Aerosol Droplets from a Mask Insert Using Mock Samples

This Example develops fundamental aspects of the mask collection strategy using simplified systems to test recovery of biomarkers from a mask insert. In these studies, direct pipetting instead of aerosol delivery, is used to investigate what the best insert material in terms of recovery from deposition. Studies are conducted on the chemistry for extraction from the paper inserts, the volume reduction that might be necessary with this process and the design the RT PCR reagents to detect the extracted material. Exhalation studies are then conducted using materials collected from non-virus infected normal human subjects and focusing on measuring the exhaled bacteria present in normal exhaled breath (Greene, V W., et al. (1962) J Bacteriol 83:663-667) by targeting bacteria thought to be resident in normal oral cavity (Aas, J A., et al. (2005) J Clin Microbiol 43:5721-5732) expected to be detectable by PCR. Measurements of oral cavity flora are also included as a “sufficient sample” control for use in single mask optimization. As part of these studies the effect of exhalation maneuvers on the exhaled material recoverable from the mask is investigated. A secondary aspect of these studies is determination of the bio safety procedures necessary to inactivate biological materials so that they may be handled safely.

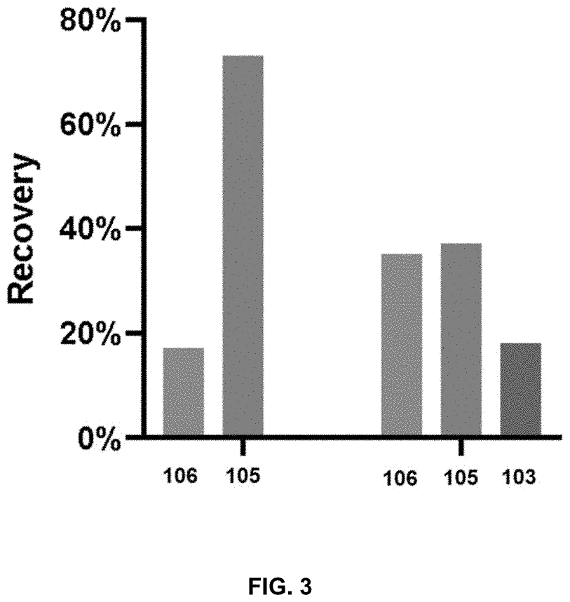

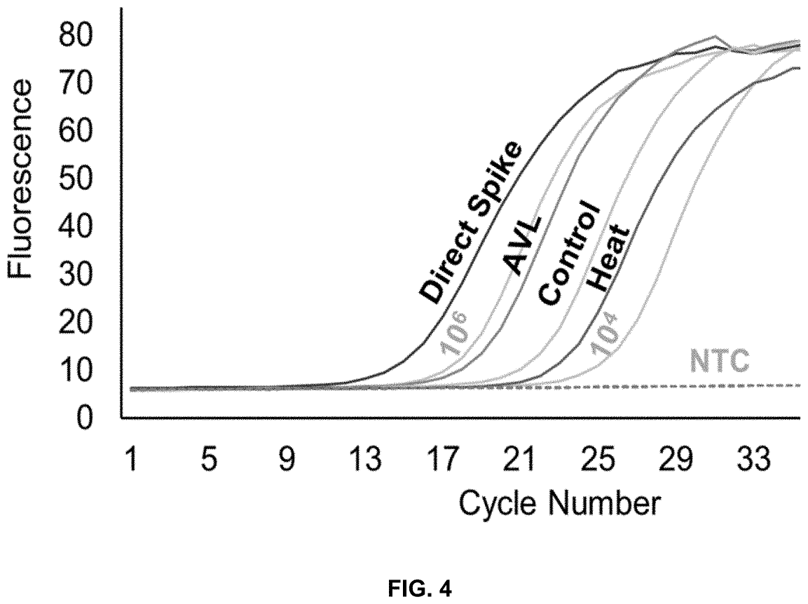

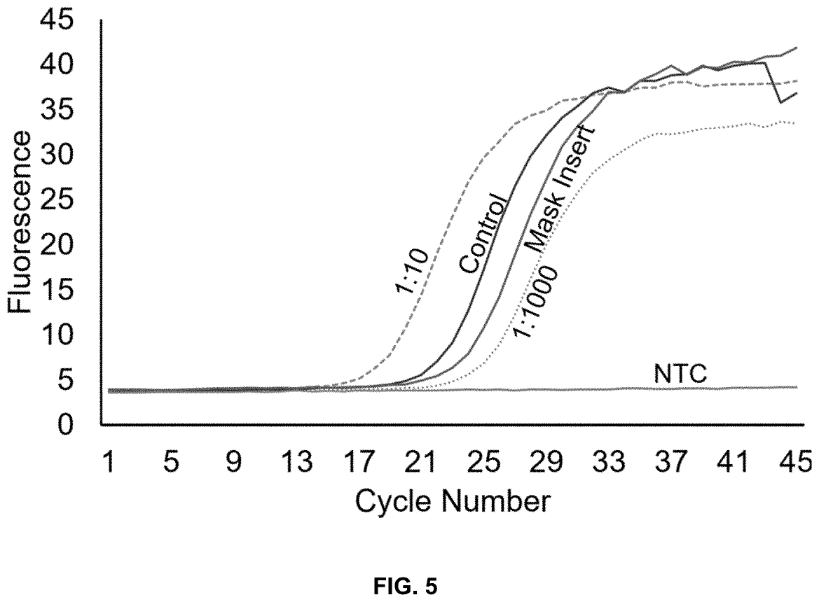

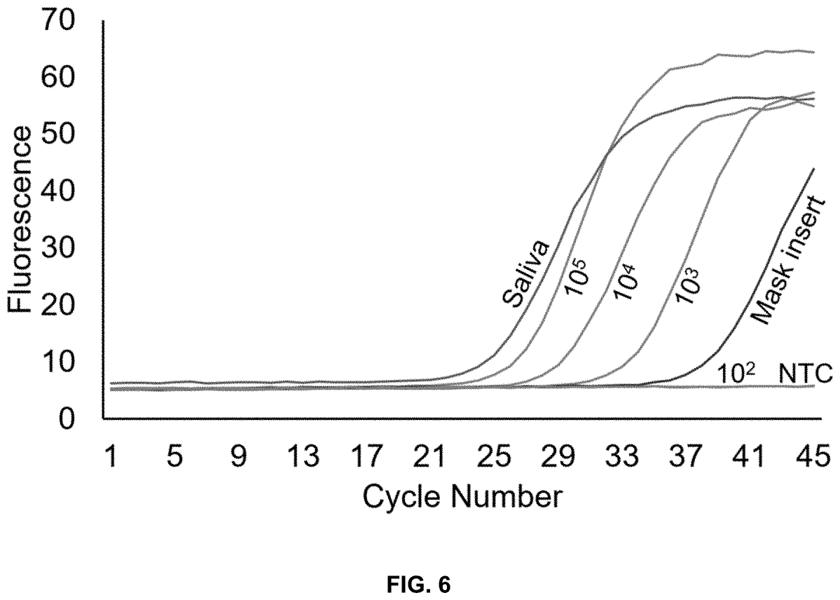

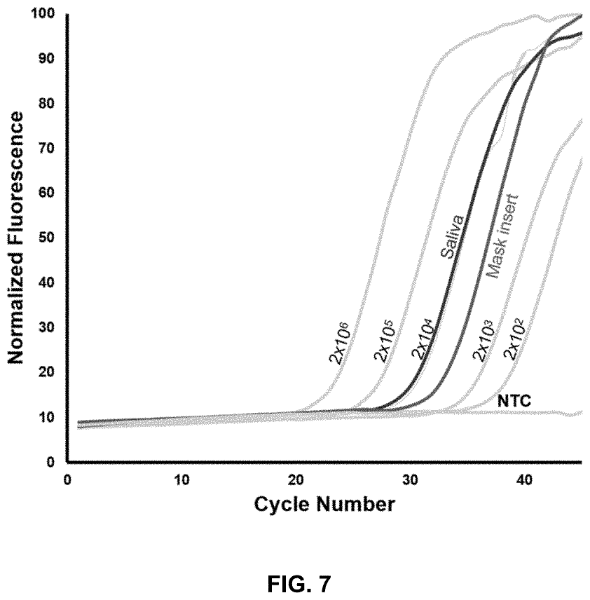

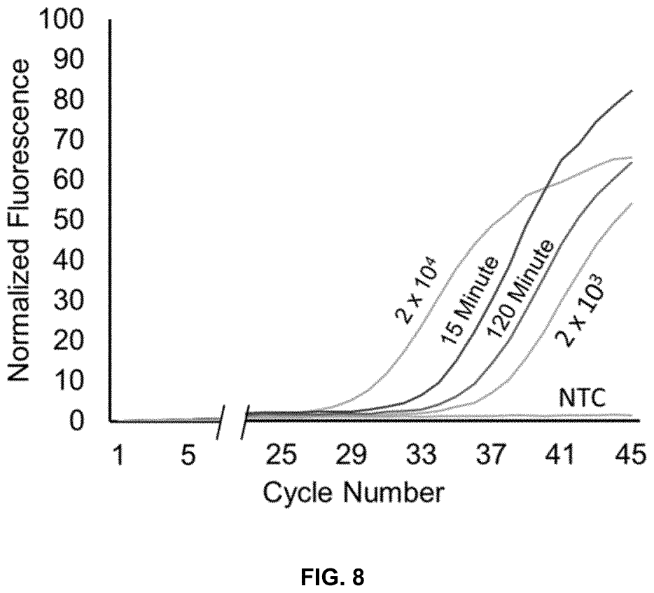

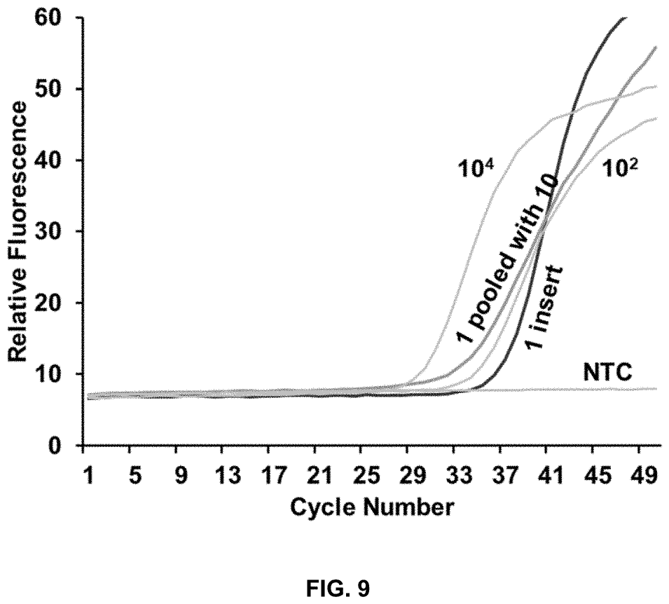

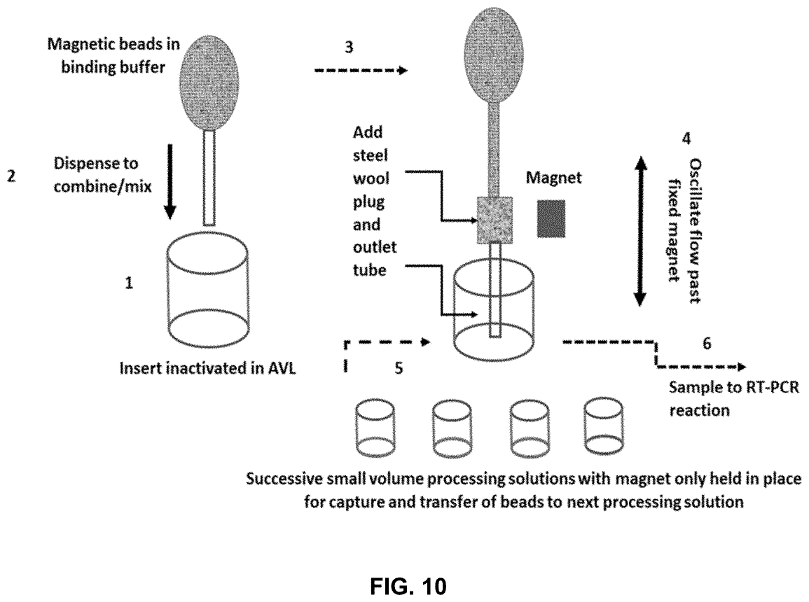

Direct pipetting tests. This project focuses on applying an insert inside of the mask instead of working with the mask material itself. This has the advantage of being optimized around a single material making the approach compatible with any type of face mask. There are a number of potential materials to investigate. To best mimic the challenges anticipated in absorption and recovery in real samples, irradiated infected cell lysate from BEI Resources (NR-52287) (from 10³ to 10⁶ copies) was applied onto the filter papers. A standard Qiagen RNeasy mini kit (Qiagen, 7104) was used to recovery RNA and this was quantified using quantified using RT-PCR. Whatman papers worked well with recovery easily detectable for even relatively low copy numbers of virus material FIG. 3. Based on positive controls with equivalent volumes of starting material (without paper), most of the RNA (about 90%) was lost during the extraction procedure. The loss due to paper-based extraction was a much smaller fraction. Based on available literature and the recent TB report (Williams, C M., et al. (2020) Lancet Infect Dis 20:607-617), this design is repeated with polyvinyl alcohol strips. The long-term advantage of this material is that it can be dissolved during the RNA extraction process and thus can help in the processing of a group of paper inserts simultaneously which is part of the overall pooling design. Other important features of a paper insert are the loading capacity and it's flow-through resistance (Chen, C C., et al. (1992) Am J Infect Control 20:177-184). In an initial test, the CDC N1 primer and probe set were used to detect irradiated viral RNA. Usually this is also combined with a second primer set in the CD standard diagnostic. Rather than using multiple reactions that the CDC kit requires, single reaction primer sets that have recently been reported to be effective (Corman, V M., et al. (2020) Euro Surveil) 25; Pfefferle, S., et al. (2020) Euro Surveill 25) are used instead. These studies focus on optimizing the fractional recovery and the limit of detection, both of which can be quantified by RT-PCR and statistically compared by an ANOVA with Bonferroni correction to determine significant changes. In addition, the pooled sampling techniques are developed using this simple direct pipetting method. In these studies, it is determined if mask insert containing a sample of irradiated virus can be detected when processed with n other samples. The goal is to determine how this impacts the limit of detection for the material spotted onto one but processed with n additional inserts. Depending on the prevalence, a higher n, the ratio of pooling test cost to individual test is given by ((n+1)/n)−(1−p)ⁿ where n is the size of the group and p is the prevalence (Dorfman, R. (1943) Ann Math Stat 14:436-440). The effect of pooling is evaluated by group testing of one positive with between 10 and 1000 negatives.

Exhalation collection studies using mock samples. Unlike the direct pipetting studies, in the exhalation studies it is important to maximize the capture of exhaled aerosol droplets in order to maximize the recovery of virus RNA from the mask. Factors that are expected to affect this include properties of the collection material itself, e.g. how much it absorbs per gram of material, the total surface area, how many inserts, what kind of air flow characteristics the insert material has and how does the air flow through the mask insert match the surrounding air flow characteristics of the surgical mask. These later factors may potentially reduce the flow of the exhaled breath through the mask insert and therefore lower the recovery of aerosol droplets containing virus. In addition, it is also clear that the entrainment of aerosols by the passage of air through the respiratory tract may be an important factor. For example, maximal entrainment is expected for sneezing and coughing, but entrainment still appears to occur during speaking and even normal breathing. Early studies of mask designs used cultured droplets to measure mask effectiveness during exhalation by speaking phrases such as “sing and chew” at 10-sec intervals for 1 min (Greene, V W., et al. (1962) J Bacteriol 83:663-667). The impact of these factors is determined using detection of resident bacteria in the oral cavity as a proxy for SAR-CoV-2. Exhaled breath from normal healthy volunteers contains bacteria that has been entrained in the exhaled breath similar to microorganisms that cause respiratory illness (Yan, J., et al. (2018) Proc Natl Acad Sci USA 115:1081-1086; Fabian, P., et al. (2008) PLoS One 3:e2691; Zheng, Y., et al. (2018) J Aerosol Sci 117:224-234). There are more than 700 bacterial species in the oral cavity with S. [Streptococcus] mitis the most commonly found species in essentially all sites and subjects (Aas, J A., et al. (2005) J Clin Microbiol 43:5721-5732). There are a number of primer sets that appear to be viable. Here Forward primer: Smi168F:5′-GAGTCCTGCATCAGCCAAGAG-3′ (SEQ ID NO:1), Reverse primer: Smi263R: 5′-GGATCCACCTTTTCTGCTTGAC-3′ (SEQ ID NO:2), Probe: Smi201T: 5′-FAM-TGTTCCCAAGTGGAGCCAACCAAACT-BHQ1-3′ (SEQ ID NO:3) (Suzuki, N., et al. J Clin Microbiol 42, 3827-3830) are used. Normal breathing, speaking, singing, coughing, sneezing are all expected to contribute to detectable exhaled material. A determination is made as to which of these produces the most by asking subjects to perform each maneuver and compare by RT-PCR the output of detectable S. mitis. The goal is to determine how long does it take to collect detectable S. mitis during normal breathing. An important part of these studies is to determine an appropriate method for reducing the bioactivity of the collected sample while maintaining detection sensitivity. In some cases, RNA recovery is assessed after a 10 minute 95° C. heat step to inactivate both bacterial and viral activity by culture. The impact on RNA recovery is determined by inoculating a mask insert with a known concentration of RNA and using quantitation RT-PCR to measure the loss.

Sufficient sample control. For evaluating one individual's infectivity, it is important to incorporate a “sufficient sample” control. Verification of S. miti in exhaled breath described above allows use of the simultaneous level of detection of this bacteria as a means to verify that sufficient biomaterial has been collected during the time that the mask was worn. Sample control is used in RT-PCR studies to verify that a negative test is not due to insufficient exhaled passing through the mask. Simultaneous measurement of the control bacteria and the SARS-CoV-2 target is done in a multiplex RT-PCR reaction by simply using different fluorescent probes. Samples for which S. mitis and SARS-Cov-2 are not detected are considered inconclusive. For pooled testing, a less quantitative colorimetric indicator is developed based on water vapor exposure or CO₂ exposure as a means to visually determine if a mask has been worn for a sufficient time. To reduce interference with the biomarker-based mask inserts, this may require a separate mask insert.

### Example 2. Develop and Implement Protocols for Evaluation of Exhaled Breath as a Method for Identifying SARS-CoV-2 Infected Individuals

In this Example 2, the disclosed methods are applied to mask inserts obtained during Vanderbilt hospital admissions—including from both symptomatic and asymptomatic individuals—to answer three critical questions that address the utility of this strategy for detecting viruses such as COVID-19. These questions are addressed through the following experiments.

Overall, the study design builds on current institutional procedures for the evaluation and admission of new patients, either when COVID-19 is suspected (symptomatic testing) or not suspected (asymptomatic screening). Currently, patients with compatible signs/symptoms for COVID-19 are evaluated in a separate section of the Emergency Department, until PCR results on nasopharyngeal specimens are known several hours later. The symptomatic individuals are enrolled when positive results are returned, as flagged by investigators. Beyond just symptomatic individuals, moreover, all Medical Center admissions are screened for asymptomatic COVID-19 infection, with the Clinical Laboratory again serving as the central point of identification. Whether symptomatic or asymptomatic, potential enrollees are consented (by a licensed resident/fellow or technologist), given a mask with insert, and instructed to breath normally (˜30 min). These masks are then sealed and delivered for initial preparation and viral inactivation. Finally, the inactivated inserts are delivered for extraction and quantitative RT-PCR testing, using the protocols developed in Example 1. As COVID-negative cohort, mask inserts are likewise obtained from asymptomatic individuals who test negative by nasopharyngeal PCR.

The investigational RT-PCR results from mask inserts are correlated with the clinical-use COVID-19 results from nasopharyngeal specimens. Excluding samples with insufficient insert material, and using the diagnostic test as the comparator, the positive agreement (sensitivity) and negative agreement (specificity) of the exhaled aerosol testing are calculated. In order to demonstrate agreement with a lower confidence-bounds >90% (for both symptomatic and asymptomatic cohorts), at least 50 COVID-19 positive individuals from each group are analyzed, which should be more than obtainable over the course of study (given current and projected future local prevalence estimates). False negatives are examined for exclusion based on the sufficient sample control and retesting of mask inserts. If still negative protocols may be modified to improve the extraction efficiency, increase exhalation of aerosols using the speaking protocols discussed in alternative designs in Example 1, the paper area used to extract could be increased, or the length of time the mask is worn could be increased. Mask inserts that are false positives are also re-tested to exclude workflow contamination and further evaluated as part of the following question.

Throughout the course of each patient's hospitalization, vital signs including respiratory rate, oxygen saturation, and temperature are measured and recorded at standard intervals. If a mask insert tests positive and the hospitalization test reports negative (mask false positive), the insert is retested using an additional sample of the stored insert material. If this also reports a positive result, the additional medical records are accessed to determine if additional COVID-19 testing was performed and, if not, notify the treating clinical team and request that additional testing be performed. If a subsequent clinical testing for SARS-CoV-2 is positive, that is interpreted as an asymptomatic or pre-symptomatic individual who was corrected identified by the mask insert at the time of hospitalization.

If a mask insert tests positive and the ER admission test reports negative (false positive), the insert is retested using an additional sample of the stored insert material. If this also reports a positive result, the additional medical records are accessed to determine if additional COVID-19 testing was performed and, if not, request that additional testing be performed. If a subsequent COVID-19 test returns a positive indication, that is interpreted as an asymptomatic or pre-symptomatic individual that was correctly identified during the ER admission testing

To answer whether the pooled mask detection method successfully detects an infected individual when tested with a group of uninfected individuals, stored insert materials left over from the initial mask insert testing are used. Based on the initially pooling limitations protocols developed in Example 1, one known positive paper insert is combined with a large number of negative insert materials, RNA extracted from the group and RT-PCR performed on the pooled sample. This can be performed when a large number of mask insert materials have been collected.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A kit for viral surveillance of essential workers, the kit comprising:
filter paper inserts configured to attach or adhere to the inside of a face mask;
extraction buffers configured to extract virion RNA from the filter paper inserts;
reagents for reverse transcription of the virion RNA into cDNA; and
oligonucleotide primers and/or probes configured to assay for the presence of a respiratory virus cDNA.

2. The kit of claim 1, wherein the filter paper insert is a water-soluble fabric.

3. The kit of claim 2, wherein the filter paper insert comprises a polyvinyl alcohol (PVA) fabric.

4. The kit of claim 1, further comprising oligonucleotide primers and/or probes configured to assay for the presence of a bacteria resident in normal oral cavity.

5. The kit of claim 1, wherein the respiratory virus comprises a influenza virus, respiratory syncytial virus, parainfluenza viruses, metapneumovirus, rhinovirus, coronaviruses, adenoviruses, or bocaviruses.

6. A surveillance face mask comprising one or more removable filter paper inserts attached or adhered to the inside of the face mask.

7. The surveillance face mask of claim 5, wherein the filter paper insert is a water-soluble fabric.

8. The surveillance face mask of claim 6, wherein the filter paper insert comprises a polyvinyl alcohol (PVA) fabric.

9. A method for surveillance of a group of subjects, the method comprising:
(a) providing face masks to the group of subjects, the face masks comprising filter paper inserts attached or adhered to the inside of the face masks;
(b) removing the filter paper inserts from the face masks after use and pooling them together; and
(c) assaying the pooled filter paper inserts for respiratory virus RNA.

10. The method of claim 9, wherein the respiratory virus comprises a influenza virus, respiratory syncytial virus, parainfluenza viruses, metapneumovirus, rhinovirus, coronaviruses, adenoviruses, or bocaviruses.

11. The method of claim 9, further comprising assaying the filter paper inserts for a bacteria resident in normal oral cavity.

12. The method of claim 11, wherein the bacteria is Streptococcus mitis.

13. The method of claim 9, wherein the subjects are essential workers.

14. The method of claim 9, further comprising screening the group of subjects for infection if respiratory virus RNA is detected in the pooled filter paper inserts.
